(19) United States
(12) Patent Application Publication
(10) Pub. No.: US 2013/0029421 A1
(43) Pub. Date: Jan. 31, 2013

(54) Methods for Making Cell Culture Substrates

(71) Applicants: Kyriakos Komvopoulos, Orinda, CA (US); Qian Cheng, Albany, CA (US)

(72) Inventors: Kyriakos Komvopoulos, Orinda, CA (US); Qian Cheng, Albany, CA (US)

(21) Appl. No.: 13/559,369

(22) Filed: Jul. 26, 2012

Related U.S. Application Data
(60) Provisional application No. 61/511,937, filed on Jul. 26, 2011.

(51) Int. Cl. C12N 5/07; B05D 7/00; B05D 3/10

(52) U.S. Cl. 435/396; 435/402; 427/535

(57) ABSTRACT

Cell culture substrates are provided. Aspects of the cell culture substrate include a substrate with a surface having at least one hydrophilic region and at least one hydrophobic region, and a surfactant layer present on the surface of the substrate and configured to produce a cell-binding surface on the hydrophilic regions of the surface. Also provided are kits which include the cell culture substrate, as well as methods of producing the cell culture substrate. The cell culture substrate and methods described herein find use in a variety of applications, including single-cell culture applications.

### CROSS-REFERENCE TO RELATED APPLICATION

Pursuant to 35 U.S.C. §119(e), this application claims priority to the filing date of U.S. Provisional Patent Application Ser. No. 61/511,937, filed Jul. 26, 2011, the disclosure of which is herein incorporated by reference.

### REFERENCE TO GOVERNMENT SUPPORT

This invention was made with government support under Grant Number CMS-0528506 awarded by the National Science Foundation. The government has certain rights in the invention.

### INTRODUCTION

Cell culture is a process by which cells are grown under controlled conditions. In practice, cell culturing can be applied to cells derived from multicellular eukaryotes, such as animal cells, plants, fungi and microbes, including viruses, bacteria and protists. Various chemical patterning methods that produce surface patterns have been used for cell culture substrates. Contact printing and membrane-based patterning for cell culture can be accomplished by physically transferring a pattern of an external cell matrix protein to a surface and then covering surrounding areas with non-fouling molecules. Traditional photolithography methods have been used for cell patterning; for instance, surface patterning of coatings using a photoresist as the lift-off mask or surface patterning of different materials for selective molecular adsorption. In other patterning methods, surface coatings are first deposited to the substrate to provide non-fouling characteristics and cell adhesive areas are then formed on the surface coating. Traditional photolithography used in previous studies to produce surface patterns for cell culture cannot be directly applied to cell culture dishes because it requires a clean-room facility and chemicals that are harmful to cells.

### SUMMARY

Cell culture substrates are provided. Aspects of the cell culture substrate include a substrate with a surface having at least one hydrophilic region and at least one hydrophobic region, and a surfactant layer present on the surface of the substrate and configured to produce a cell-binding surface on the hydrophilic regions of the surface.

Also provided are kits which include the cell culture substrate, as well as methods of producing the cell culture substrate. The cell culture substrate and methods described herein find use in a variety of applications, including single-cell culture applications.

### BRIEF DESCRIPTION OF THE FIGURES

FIG. 1 shows images of mesenchymal stem cells (MSCs) seeded on chemically different Parylene C surfaces: (a) untreated (e.g., hydrophobic), (b) oxygen plasma-treated (e.g., hydrophilic), (c) untreated and incubated with 0.1% Pluronic F108 solution, and (d) oxygen plasma-treated and incubated with 0.1% Pluronic F108 solution, according to embodiments of the present disclosure.

FIG. 2 shows images of MSCs on chemically patterned Parylene C surfaces incubated with different solutions: (a) 0.01% Pluronic F108, (b) 0.1% Pluronic F108, (c) 0.01% Pluronic F108 mixed with 25 μg/mL fibronectin, (d) 0.1% Pluronic F108 mixed with 25 μg/mL fibronectin, and (e) 0.01% Pluronic F108 followed by incubation with 10% serum medium, according to embodiments of the present disclosure. Statistical results of cell patterning yield corresponding to incubation with solutions (a), (d), and (e) are compared in the graph shown in FIG. 2(f).

FIG. 3 shows schematic drawings of (a-c) surface chemical patterning process, (d) surface incubation with Pluronic F108 solution, (e) surface activation by serum proteins, and (f) selective attachment of single cells on hydrophilic surface areas, according to embodiments of the present disclosure. FIG. 3(g) shows a fluorescence photograph showing FITC-collagen adsorption on a chemically patterned Parylene C surface incubated with 0.01% Pluronic F108 solution, according to embodiments of the present disclosure.

FIG. 4 shows images of MSCs seeded on plasma-modified polystyrene (PS) surfaces treated with different solutions after incubation with (a)-(f) plain DMEM or (g)-(l) serum medium for 4 hr, according to embodiments of the present disclosure.

FIG. 5 shows images of MSCs seeded on patterned PS surfaces treated with different solutions after incubation with serum medium overnight: (a) untreated (control), (b) PBS, (c) 0.01% Pluronic F108, (d) 1% Pluronic F108, (e) 0.01% Pluronic F108 and 25 μg/mL of fibronectin, and (f) 1% Pluronic F108 and 25 μg/mL of fibronectin, according to embodiments of the present disclosure.

FIG. 6 shows graphs of C1s core level XPS peak of different PS surfaces: (a) untreated (control) PS incubated with PBS, (b) oxygen plasma-treated PS incubated with PBS, (c) untreated PS incubated with 1% Pluronic F108 solution, and (d) oxygen plasma-treated PS incubated with 1% Pluronic F108 solution, according to embodiments of the present disclosure.

FIG. 7 shows a graph of nitrogen concentration of untreated and oxygen plasma-treated PS surfaces incubated with Pluronic F108 solutions of different concentrations containing 25 μg/mL of fibronectin, according to embodiments of the present disclosure. A statistically significant difference (P<0.05) between specified groups is indicated by an asterisk.

FIG. 8 shows images of single MSCs cultured on patterned PS surfaces treated with 1% Pluronic F108 solution containing 25 μg/mL of fibronectin after incubation with serum medium for 2 weeks, according to embodiments of the present disclosure. Each pattern area is 2000 μm², while the pattern shape index is equal to (a) 1.0, (b) 0.5, (c) 0.25, and (d) 0.1.

FIG. 9 shows images of nuclei and actin staining of MSCs cultured on patterned PS surfaces after incubation with serum medium for 2 weeks, according to embodiments of the present disclosure. Each pattern area is 2000 μm², while the pattern shape index is equal to (a) 1.0, (b) 0.5, (c) 0.25, and (d) 0.1.

FIG. 10 shows graphs of the effect of surface patterning on (a) cell spreading area, (b) cell shape index (CSI), (c) nucleus projection area, and (d) nucleus shape index (NSI) of MSCs cultured on patterned PS surfaces after incubation with serum medium for 2 weeks, according to embodiments of the present disclosure. Statistically significant differences (P<0.05) compared to all other groups (10-20 cells per group) are indicated by an asterisk.

### DETAILED DESCRIPTION

Cell culture substrates are provided. Aspects of the cell culture substrate include a substrate with a surface having at least one hydrophilic region and at least one hydrophobic region, and a surfactant layer present on the surface of the substrate and configured to produce a cell-binding surface on the hydrophilic regions of the surface. Also provided are kits which include the cell culture substrate, as well as methods of producing the cell culture substrate. The cell culture substrate and methods described herein find use in a variety of applications, including single-cell culture applications.

Before the present invention is described in greater detail, it is to be understood that aspects of the present disclosure are not limited to the particular embodiments described, and as such may, of course, vary. It is also to be understood that the terminology used herein is for the purpose of describing particular embodiments only, and is not intended to be limiting, since the scope of embodiments of the present disclosure will be defined only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within embodiments of the present disclosure. The upper and lower limits of these smaller ranges may independently be included in the smaller ranges and are also encompassed within embodiments of the present disclosure, subject to any specifically excluded limit in the stated range. Where the stated range includes one or both of the limits, ranges excluding either or both of those included limits are also included in embodiments of the present disclosure.

Unless defined otherwise, all technical and scientific terms used herein have the same meaning as commonly understood by one of ordinary skill in the art to which this invention belongs. Although any methods and materials similar or equivalent to those described herein can also be used in the practice or testing of embodiments of the present disclosure, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that embodiments of the present disclosure are not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various aspects of embodiments of the present disclosure, aspects of embodiments of the cell culture substrates are described first in greater detail. Following this description, methods of making the cell culture substrates, followed by a description of embodiments for kits that include the cell culture substrates. Finally, a review of the various applications in which the cell culture substrates, methods, and kits may find use is provided.

Cell Culture Substrates

Embodiments of the present disclosure provide for a cell culture substrate. The cell culture substrate may be configured to facilitate the growth of cells on the surface of the cell culture substrate. In some embodiments, the cells may be grown under controlled (e.g., laboratory) conditions. In certain cases, the cell culture substrate includes a surface configured to facilitate attachment of one or more cells to the surface of the substrate. In some instances, attachment of the cells to the surface of the cell culture substrate may facilitate the growth of the cells on the surface of the cell culture substrate. In certain cases, attachment of the cells to the surface of the cell culture substrate may facilitate separation of cells attached to the surface from cells that are not attached to the surface of the cell culture substrate.

Cells may be attached to the surface of the cell culture substrate by various methods. In certain embodiments, the cell culture substrate includes a substrate with at least one region (e.g., a first region) configured to facilitate attachment of one or more cells to the surface of the substrate and at least one region (e.g., a second region) to which substantially no cells attach. In some cases, the first region has different physical and/or chemical properties than the second region or the surface of the cell culture substrate. In certain instances, the first and second regions have different physical properties, such as, but not limited to, hydrophilicity, hydrophobicity, contact angle, surface roughness, etc. In some cases, the first and second regions have different chemical properties, such as, but not limited to, the elemental composition, the % composition of one or more elements that the substrate is composed of, and the like.

In certain embodiments, the cell culture substrate includes a substrate with regions having different physical properties. For example, the cell culture substrate may include a substrate that has a surface with at least one hydrophilic region and at least one hydrophobic region. By “hydrophilic” is meant a substance that has a relatively strong affinity for water. By “hydrophobic” is meant a substance that has a relatively low affinity for water. In certain instances, the degree of hydrophilicity (or hydrophobicity) of a surface may be measured by measuring the contact angle of the surface. The contact angle is the angle, measured through the liquid, between a surface and a line tangent to the point at which the edge of a drop of liquid (e.g., water) meets the surface. In some cases, the hydrophilic region of the surface of the cell culture substrate has a contact angle less than 90°. For example, the hydrophilic region may have a contact angle of 80° or less, such as 70° or less, including 60° or less, or 50° or less, or 40° or less, or 30° or less, or 20° or less, or 10° or less, or 5° or less, or 1° or less. In some instances, the hydrophobic region of the surface of the cell culture substrate has a contact angle greater than 90°. For instance, the hydrophobic region may have a contact angle of 100° or more, such as 110° or more, including 120° or more, or 130° or more, or 140° or more, or 150° or more, or 160° or more, or 170° or more, or 175° or more, or 179° or more. In some instances, the contact angle may be measured using the drop shape method, as described in more detail by Roger P. Woodward, Ph.D., Contact Angle Measurements Using the Drop Shape Method, Portsmouth, Va.: First Ten Angstroms.

In certain embodiments, the cell culture substrate includes a substrate with regions having different chemical properties. For instance, the hydrophilic region of the substrate may include a compound having different functional groups than the hydrophobic region of the substrate. In some cases, the hydrophilic region of the substrate may have a different percent composition of one or more functional groups than the hydrophobic region of the substrate. For example, the hydrophilic region may have a greater amount of oxygen-containing functional groups than the hydrophobic region. In some instances, the amount of oxygen-containing functional groups is measured as the oxygen/carbon (O/C) ratio by X-ray photoelectron spectroscopy (XPS). In certain cases, the hydrophilic region may have a greater O/C ratio than the hydrophobic region. In some cases, the hydrophilic region has an O/C ratio of 0.25 or more, such as 0.3 or more, including 0.35 or more, or 0.4 or more, or 0.45 or more, or 0.5 or more, or 0.55 or more, or 0.6 or more, or 0.65 or more, or 0.7 or more, or 0.75 or more. In some instances, the hydrophilic region has an O/C ratio of 0.4 or more, such as 0.5. In some cases, the hydrophobic region has an O/C ratio of 0.4 or less, such as 0.35 or less, including 0.3 or less, or 0.25 or less, or 0.2 or less, or 0.15 or less, or 0.1 or less, or 0.05 or less. In some instances, the hydrophobic region has an O/C ratio of 0.35 or less, such as 0.25 or less.

The substrate may be in the form of a plate, a tube, sphere, or complex shape. In some embodiments, the substrate is in the form of a plate (e.g., a cell culture plate) or a dish (e.g., a Petri dish) so that the formed article can be used directly as a cell culture substrate. In certain instances, the surface of the substrate is substantially planar. The surface of the substrate may also have a convex shape, a concave shape, or other suitable shapes for cell culture substrates.

In certain embodiments, the substrate is a polymeric substrate. Any suitable polymeric substrate may be used in embodiments of the cell culture substrate. The polymeric substrate may include any suitable polymer including, but not limited to, homopolymers, copolymers, combinations thereof, and the like. Examples of suitable polymers include, but are not limited to, polystyrene, polyethylene terephthalate (PET), polypropylene, polyethylene, polymethylmethacrylate, silicone, polyurethane, combinations thereof, and the like. In some cases, the polymeric substrate includes polystyrene. In certain instances, the polymeric substrate has a thickness of 5 mm or less, such as 4 mm or less, including 3 mm or less, or 2 mm or less, or 1 mm or less.

In some embodiments, the substrate is a polymeric layer on the surface of a non-polymeric support. For example, the non-polymeric support may include glass. In some instances, the polymeric layer may include, but is not limited to, a poly(p-xylylene), polystyrene, polyethylene terephthalate (PET), polypropylene, polyethylene, polymethylmethacrylate, silicone, polyurethane, combinations thereof, and the like. In certain cases, the polymeric layer includes poly(p-xylylene). In certain instances, the polymeric layer has a thickness of 1 mm or less, such as 500 μm or less, including 250 μm or less, or 100 μm or less, or 50 μm or less, or 25 μm or less, or 10 μm or less, or 5 μm or less, or 1 μm or less, or 0.5 μm or less, or 0.1 μm or less. In some embodiments, the polymeric layer has a thickness of 0.5 μm.

Embodiments of the cell culture substrate include a surfactant layer present on at least a portion of the surface of the substrate. In certain embodiments, the surfactant layer is present on substantially the entire surface of the substrate. For instance, the surfactant layer may be present on the hydrophilic regions and the hydrophobic regions of the substrate. In other embodiments, the surfactant layer is present on a portion of the substrate. For instance, the surfactant layer may be present on the hydrophobic regions of the substrate. In other cases, the surfactant layer is present on the hydrophilic regions of the substrate. The surfactant layer may be attached to the surface of the substrate. In certain instances, the surfactant layer is attached to the surface of the substrate through covalent bonds. In other cases, the surfactant layer is attached to the surface of the substrate through non-covalent interactions, such as, but not limited to, ionic bonds, hydrophobic interactions, hydrogen bonds, van der Waals forces (e.g., London dispersion forces), dipole-dipole interactions, and the like.

In certain embodiments, the surfactant layer is configured to produce a cell-binding surface on the surface of the substrate. The cell-binding surface may be configured to facilitate attachment of one or more cells to the surface of the substrate. The cell-binding surface may be present on at least a portion of the surface of the substrate. In certain embodiments, the cell-binding surface is present on substantially the entire surface of the substrate. In these embodiments, the cell-binding surface may facilitate attachment of one or more cells over substantially the entire surface of the substrate. In other embodiments, the cell-binding surface is present on a portion of the substrate. In these embodiments, the cell-binding surface may facilitate attachment of one or more cells on the portions of the surface of the substrate where the cell-binding surface is present. For instance, the cell-binding surface may be present on the hydrophilic regions of the substrate, and thus may facilitate attachment of one or more cells to the hydrophilic regions of the substrate.

In some cases, the surfactant layer is present on substantially the entire surface of the substrate but only forms a cell-binding surface on the hydrophilic regions of the substrate. For example, the physical properties of the surfactant layer may depend on the physical and/or chemical properties of the underlying substrate surface. In some embodiments, the physical properties of the surfactant layer depend on the hydrophilicity of the underlying substrate surface. The hydrophilic regions of the substrate surface may modify the physical properties of the surfactant layer to form the cell-binding surface on the hydrophilic regions of the substrate surface, and thus facilitate attachment of one or more cells to the hydrophilic regions of the substrate. For instance, the conformation the surfactant layer may depend on the hydrophilicity of the substrate surface. If the substrate surface is hydrophilic, the surfactant layer may have a conformation that facilitates attachment of one or more cells to the hydrophilic regions of the substrate. In some cases, the conformation of the surfactant layer on the hydrophilic regions of the substrate surface minimizes steric hindrances between the substrate surface and the cells. A minimization in steric hindrances may facilitate attachment of the cells to the substrate surface. In certain instances, for surfactants that include an elongated portion as described in more detail below, the surfactants in the surfactant layer may have a conformation such that the elongated portion of the surfactant is positioned proximate to the substrate surface along the length of the surfactant.

In certain embodiments, the hydrophobic regions of the substrate surface may modify the physical properties of the surfactant layer to reduce the occurrence of attachment of cells to the hydrophobic regions of the substrate. For instance, the hydrophobic regions of the substrate surface may modify the physical properties of the surfactant layer such that substantially no cells attach to the hydrophobic regions of the substrate. In some cases, as described above, the conformation the surfactant layer may depend on the hydrophilicity of the substrate surface. If the substrate surface is hydrophobic, the surfactant layer may have a conformation that reduces the occurrence of attachment of cells to the hydrophobic regions of the substrate. In some cases, the conformation of the surfactant layer on the hydrophobic regions of the substrate surface maximizes steric hindrances between the substrate surface and the cells. Maximization of steric hindrances may reduce the occurrence of attachment of the cells to the substrate surface. In certain instances, for surfactants that include an elongated portion as described in more detail below, the surfactants in the surfactant layer may have a conformation such that the elongated portion of the surfactant extends away from the surface of the substrate. In these instances, the binding interactions between the cells and the substrate surface may be minimized.

The surfactant layer includes a surfactant. A surfactant is a compound that is amphiphilic, such that it contains one or more hydrophobic groups and one or more hydrophilic groups. In some instances, the hydrophilic groups of the surfactant may be elongated. Surfactants that include an elongated hydrophilic portion may have a conformation such that the elongated hydrophilic portion of the surfactant is positioned proximate to the substrate surface along the length of the surfactant. For example, the elongated hydrophilic portion of the surfactant may be attracted to the hydrophilic region of the substrate surface and adopt a conformation where the elongated hydrophilic portion of the surfactant lies proximate to the substrate surface (e.g., does not substantially extend away from the substrate surface). In these embodiments, steric hindrances between the substrate surface and cells may be minimized, thus facilitating attachment of cells to the hydrophilic regions of the substrate. Conversely, the elongated hydrophilic portion of the surfactant may be repelled by the hydrophobic regions of the substrate surface and adopt a conformation where the elongated hydrophilic portion of the surfactant extends away from the surface of the substrate. In these embodiments, steric hindrances between the substrate surface and cells may be maximized, thus reducing the occurrence of attachment of cells to the hydrophobic regions of the substrate.

In certain embodiments, the same surfactant may adopt different conformations on different regions of the substrate surface. As described above, the surfactant may have a configuration where the elongated hydrophilic portion of the surfactant is attracted to the hydrophilic region of the substrate surface and does not substantially extend away from the substrate surface. In addition, the surfactant may have a configuration where the elongated hydrophilic portion of the surfactant is repelled by the hydrophobic region of the substrate surface and extends away from the substrate surface. As such, a surfactant layer present on substantially the entire surface of the substrate may produce cell-binding surfaces on only the hydrophilic regions of the surface of the substrate, as described above.

In certain embodiments, the surfactant includes a copolymer of hydrophilic and hydrophobic monomers. In some cases, the surfactant includes a copolymer of polyalkylene oxides. Suitable polyalkylene oxides include, but are not limited to, polyethylene oxide (PEO, also known as polyethylene glycol (PEG) or polyoxyethylene (POE)), polypropylene oxide (PPO, also known as polypropylene glycol), combinations thereof, and the like. For example, the surfactant may include a copolymer of a hydrophilic polyalkylene oxide (e.g., PEO) and a hydrophobic polyalkylene oxide (e.g., PPO). In certain instances, the surfactant has a molecular weight of 1000 or more, such as 2000 or more, including 3000 or more, or 5000 or more, or 8000 or more, or 10,000 or more, or 15,000 or more, or 20,000 or more, etc. In certain embodiments, the surfactant has a molecular weight of 3000. In some instances, the surfactant includes a content of PEO of 10% or more, such as 20% or more, including 30% or more, or 40% or more, or 50% or more, or 60% or more, or 70% or more, or 80% or more, or 90% or more. In certain embodiments, the surfactant includes 80% PEO.

Suitable surfactants include, but are not limited to, Pluronic F108 (MW 3000, 80% PEO), Pluronic F68 (MW 1800, 80% PEO), Pluronic F127 (MW 3600, 70% PEO), PEG 8,000 and PEG 20,000, sodium dodecyl sulfate, sodium dioctyl sulfosuccinate, polysorbate 80, polyoxyl 40 hydrogenated castor oil, combinations thereof, and the like. In certain embodiments, the surfactant includes a copolymer of PEO and PPO with a molecular weight of 3000 and 80% PEO content (e.g., Pluronic F108).

In certain embodiments, the cell culture substrate further includes a cell-binding agent configured to facilitate attachment of cells to the substrate surface. In some instances, the cell-binding agent is a proteinaceous cell-binding agent on the cell-binding surface of the substrate. By “proteinaceous” is meant that the cell-binding agent includes one or more proteins or peptides, or derivatives thereof. The proteinaceous cell-binding agent may be configured to facilitate attachment of cells to the substrate surface. For example, the presence of a proteinaceous cell-binding agent on the cell-binding surface may increase the strength or attachment and/or the number of cells attached to the substrate surface. In certain cases, the proteinaceous cell-binding agent includes a protein, such as a glycoprotein. For instance, the proteinaceous cell-binding agent may include fibronectin. In some cases, the proteinaceous cell-binding agent includes serum.

In certain embodiments, the cell culture substrate further includes one or more cells disposed on the cell-binding surface. Cells can include any type of cell suitable for cell culture, such as, but not limited to, eukaryotic cells, prokaryotic cells, etc. For example, cells may include animal cells, plant cells, bacteria, fungi, and the like. In some instances, the cell culture substrate may be configured for single-cell culture, where single cells are attached to discrete regions of the cell culture substrate. In these instances, a single cell may be disposed on each cell-binding surface on the hydrophilic regions of the substrate surface. In some cases, the cell-binding surface may be sized to be approximately the same size as a single cell to facilitate single-cell cultures. For instance, the cell-binding surface may have a diameter (e.g., for cell-binding surfaces that have a round shape) of 500 μm or less, such as 400 μm or less, including 300 μm or less, or 200 μm or less, or 100 μm or less or 75 μm or less or 50 μm or less, or 25 μm or less, or 10 μm or less, or 5 μm or less, or 1 μm or less. In certain cases, the cell-binding surface may have a diameter of 25 μm or less. The hydrophilic regions of the substrate surface may be sized with dimensions similar to the cell-binding surface described above. Stated another way, in some instances, the cell-binding surface may cover substantially the entire hydrophilic region, and thus the hydrophilic region and the cell-binding surface have substantially the same dimensions.

As described above, the cell-binding surface may be formed on the hydrophilic regions of the substrate surface. In some instances, the surface of the substrate includes an array of hydrophilic regions on the substrate surface. The array of hydrophilic regions may include a plurality of distinct hydrophilic regions, such as 2 or more hydrophilic regions, including 5 or more, or 10 or more, or 25 or more, or 50 or more, or 75 or more, or 100 or more, or 250 or more, or 500 or more, or 750 or more, or 1,000 or more, or 2,000 or more, or 5,000 or more, or 10,000 or more hydrophilic regions. The distinct hydrophilic regions in the array are generally present as a pattern, where the pattern may be in the form of organized rows and columns of hydrophilic regions, e.g., a grid of hydrophilic regions, across the substrate surface, a series of curvilinear rows across the substrate surface, e.g., a series of concentric circles or semi-circles of hydrophilic regions, and the like. The density of hydrophilic regions in the array may vary, but will generally be 10 or more, including 100 or more, 250 or more, or 500 or more, or 1,000 or more, or 2,000 or more hydrophilic regions per cm².

In certain embodiments, the hydrophilic regions may be surrounded by a hydrophobic region, such that the surface of the substrate includes an array of hydrophilic regions in a hydrophobic region. For instance, the surface of the substrate may include a substantially contiguous hydrophobic region with the array of hydrophilic regions in the substantially contiguous hydrophobic region. As described above, in certain embodiments, the array of hydrophilic regions may be arranged on the surface of the substrate in a pattern. In these embodiments, the array of hydrophilic regions may be arranged in a pattern in a hydrophobic region (e.g., a substantially contiguous hydrophobic region).

In certain embodiments, the cell culture substrate is configured to provide for stable cells for an extended period of time. In some cases, the cell culture substrate is configured to provide for stable cells for 1 week or more, such as 2 weeks or more, including 3 weeks or more, or 4 weeks or more, or several months or more (e.g., 2 months, 3 months, 4 months, 5 months, 6 months, or 9 months, or 12 months or more). The cell culture substrate may be configured to be compatible with cell growth, such that the cell culture substrate does not include toxic compounds or chemicals that may interfere with cell growth. In some instances, the cell culture substrate may be substantially sterile, such that the cell culture substrate includes substantially no undesired bacteria or microbes.

Methods

Aspects of the present disclosure also include methods of producing the cell culture substrates described herein. The method for producing the cell culture substrate includes exposing a substrate to a plasma to produce a surface having at least one hydrophilic region and at least one hydrophobic region. Exposing the substrate to a plasma may physically and/or chemically modify the surface of the substrate. For example, exposing the substrate to a plasma may physically modify the surface of the substrate by increasing the hydrophilicity of the surface of the substrate exposed to the plasma. In certain embodiments, exposing the substrate to the plasma produces one or more hydrophilic regions on the substrate surface where the substrate surface has been exposed to the plasma.

In certain instances, exposing the substrate surface to the plasma produces a substrate surface with a contact angle less than 90°, such as 80° or less, or 70° or less, including 60° or less, or 50° or less, or 40° or less, or 30° or less, or 20° or less, or 10° or less, or 5° or less, or 1° or less. In some cases, the hydrophilicity of the substrate surface exposed to the plasma may be increased by increasing the % composition of one or more functional groups in the regions of the substrate surface exposed to the plasma. For instance, exposing the substrate surface to the plasma may increase to amount of oxygen-containing functional groups in the regions of the substrate surface exposed to the plasma. In some cases, exposing the substrate surface to the plasma increases the oxygen/carbon (O/C) ratio in the regions of the substrate surface exposed to the plasma.

In certain embodiments, the step of exposing the substrate to a plasma includes forming a pattern of hydrophilic regions on the surface of the substrate. In some cases, the step of exposing the substrate to plasma includes applying a mask that includes a masked portion and an unmasked portion to the surface of the substrate, contacting the masked substrate with the plasma to produce a hydrophilic region in the unmasked portion of the substrate, and removing the mask from the substrate. In some instances, the mask includes a pattern of unmasked portions, such that contacting the masked substrate with the plasma produces a pattern of hydrophilic regions on the substrate surface that corresponds to the pattern of unmasked portions in the mask. Any desired pattern of hydrophilic regions may be formed on the substrate surface. For example, a grid of rows and columns of hydrophilic regions may be formed on the substrate surface using the methods as described above.

Various types of plasma may be used to produce the hydrophilic regions on the substrate surface, such as plasma formed from any suitable precursor gas. Suitable precursor gases may include, but are not limited to, oxygen, hydrogen, nitrogen and noble gases (e.g., argon, etc.). In certain embodiments, the precursor gas is oxygen, such that the method includes exposing the substrate surface to oxygen plasma. Generally, plasma treatment of a polymeric substrate can be achieved by contacting the substrate with the gas to be used in the plasma treatment and applying high-energy radiation, sufficient to ionize the gas to a plasma state. While not intending to be bound by any particular theory, in some cases, the plasma activates the polymer chains that are in contact with the plasma by dissociating covalent bonds in the polymer chains. The reactions that then occur at these activated sites will vary with the operating conditions such as the power density, exposure time, working pressure, type of gas, gas flow rate, temperature, electrode spacing, chamber dimensions, substrate bias voltage, or combinations of these conditions. A capacitively coupled plasma (CCP) process or an inductively coupled plasma (ICP) process may be used. In certain embodiments, a power of 200 W or greater is used when exposing the substrate to the plasma, such as 300 W or greater, or 500 W or greater, or 750 W or greater, or 1000 W or greater, or 1200 W or greater, or 1500 W or greater. In some instances, the plasma has an ion energy fluence (J/m²) of 1×10¹ or more, such as 1×10² or more, including 1×10³ or more, or 1×10⁴ or more, or 1×10⁵ or more, or 1×10⁶ or more. Types and methods of plasma treatment of surfaces are also described in U.S. application Ser. Nos. 11/741,408 and 11/942,909, the disclosures of each of which are incorporated herein by reference in their entirety.

As described above, the substrate may be a polymeric substrate. In these embodiments, the polymeric substrate may be contacted directly with the plasma as described above. In other embodiments, the substrate may include a non-polymeric support. In these embodiments, the method may include depositing a polymeric layer on the surface of the support (e.g., the non-polymeric support) prior to exposing the substrate to the plasma. In some cases, the polymeric layer includes poly(p-xylylene). In certain instances, the depositing may include chemical vapor deposition (e.g., low-pressure chemical vapor deposition) of polymeric precursors onto the surface of the support to form the polymeric layer.

In certain embodiments, the method further includes contacting the surface of the substrate with a surfactant to form a surfactant layer on the surface of the substrate. In some instances, the entire substrate surface is contacted with the surfactant, such that the surfactant layer covers substantially the entire substrate surface. For instance, the surfactant layer may be disposed on the hydrophilic regions and the hydrophobic regions of the substrate surface, as described above. In other embodiments, one or more portions of the substrate surface are contacted with the surfactant, such that the surfactant is disposed substantially only on the one or more portions of the substrate surface contacted with the surfactant. For example, the surfactant may be disposed substantially only on the hydrophilic regions of the substrate surface. In certain instances, applying the surfactant layer to the surface of the substrate produces a cell-binding surface on the hydrophilic regions of the surface, as described above.

The surfactant may be provided in various concentrations. For example, the surfactant may be provided in a solution where the concentration of the surfactant is 0.001% or more, such as 0.01% or more, or 0.1% or more, including 1% or more. In certain embodiments, the concentration of the surfactant is 0.001%. In certain embodiments, the concentration of the surfactant is 0.01%. In certain embodiments, the concentration of the surfactant is 0.1%. In certain embodiments, the concentration of the surfactant is 1%.

The surfactant may be contacted with the substrate surface for a period of time sufficient to allow the surfactant to attach to the substrate surface. For instance, the surfactant may be contacted with the substrate surface for a period of time such that the surfactant attaches to the substrate surface and is not significantly removed from the substrate surface by subsequent washing of the substrate. In certain cases, the period of time sufficient to attach the surfactant to the substrate surface is 24 hours or less, including 20 hours or less, such as 16 hours or less, or 12 hours or less, or 10 hours or less, or 8 hours or less, or 6 hours or less, or 4 hours or less, or 2 hours or less. In certain embodiments, the period of time sufficient to attach the surfactant to the substrate surface is 8 hours or less. In certain embodiments, the step of contacting the surface of the substrate with the surfactant may be performed at a temperature less than ambient room temperature. For example, the step of contacting the surface of the substrate with the surfactant may be performed at a temperature of 25° C. or less, such as 20° C. or less, including 15° C. or less, or 10° C. or less, or 5° C. or less. In some cases, the step of contacting the surface of the substrate with the surfactant is performed at 4° C.

In certain instances, the method further includes removing unbound surfactant from the substrate. For example, the method may include washing unbound surfactant away from the substrate surface. The washing may include washing the substrate surface with a solution, such as a buffer solution (e.g., phosphate buffered saline). In some cases, the step of washing unbound surfactant from the cell culture substrate is performed after contacting the surfactant with the substrate surface for a sufficient amount of time to allow surfactant to attach to the surface of the substrate. In certain embodiments, washing the unbound surfactant from the substrate surface does not significantly remove surfactant attached to the substrate surface. One or more washing steps may be performed as desired.

In some instances, the method further includes placing a cell on the cell culture substrate. The cell may be disposed on the cell-binding surface on the hydrophilic regions of the substrate surface. In certain cases, disposing a cell on the cell culture substrate includes contacting the surface of the substrate with a fluid that contains cells to be deposited on the substrate surface. The fluid may be a buffer or other fluid compatible with the cells. In some instances, the fluid containing the cells is contacted with substantially the entire surface of the substrate. As described above, in some cases, the cells may only become attached to the cell-binding surface on the hydrophilic regions of the surface of the substrate, and may not significantly attach to the hydrophobic regions of the surface of the substrate. In other embodiments, the fluid containing the cells is contacted with only certain regions on the surface of the substrate. For instance, the fluid containing the cells may only be deposited on the hydrophilic regions of the surface of the substrate.

In some instances, the cells may be provided in a solution (e.g., a cell culture medium, such as a serum-free medium or serum containing medium). The cells may be provided in various concentrations suitable for cell cultures. In some cases, the concentration of the cells is 0.001% or more, such as 0.01% or more, or 0.1% or more, including 1% or more, or 5% or more, or 10% or more, or 15% or more, or 20% or more, or 25% or more.

The cells may be contacted with the cell-binding surface for a period of time sufficient to allow the cells to attach to the cell-binding surface. For instance, the cells may be contacted with the cell-binding surface for a period of time such that the cells attach to the cell-binding surface and are not significantly removed from the cell-binding surface by subsequent washing of the cell culture substrate. In certain cases, the period of time sufficient to attach the cells to the cell-binding surface is 5 hours or less, including 4 hours or less, such as 3 hours or less, or 2 hours or less, or 1 hours or less, or 45 minutes or less, or 30 minutes or less, or 15 minutes or less. In certain embodiments, the period of time sufficient to attach the cells to the cell-binding surface is 1 hour or less. In certain embodiments, the step of contacting the cell-binding surface with the cells may be performed at a temperature greater than ambient room temperature. For example, the step of contacting the cell-binding surface with the cells may be performed at a temperature of 20° C. or more, such as 25° C. or more, including 30° C. or more, or 35° C. or more, or 40° C. or more. In some cases, the step of contacting the cell-binding surface with the cells is performed at a temperature similar to human body temperature, 37° C.

In some embodiments, a single cell is disposed on each cell-binding surface. In some cases, a single cell is attached to each cell-binding surface. Methods that include attaching a single cell to each cell-binding surface may facilitate the production of single-cell cultures.

In certain instances, the method further includes removing unbound cells from the cell culture substrate. For example, the method may include washing unbound cells away from the substrate surface. The washing may include washing the substrate surface with a solution, such as a buffer solution (e.g., phosphate buffered saline). In some cases, the step of washing unbound cells from the cell culture substrate is performed after contacting the cells with the substrate surface for a sufficient amount of time to allow cells to attach to the cell-binding surface on the hydrophilic regions of the substrate surface. In certain embodiments, washing the unbound cells from the substrate surface does not significantly remove cells attached to the cell-binding surface on the hydrophilic regions of the substrate surface. One or more washing steps may be performed as desired.

In some cases, the method further includes contacting the cell-binding surface with a proteinaceous cell-binding agent prior to disposing the cell on the cell-binding surface. In certain cases, contacting the cell-binding surface with the proteinaceous cell-binding agent includes contacting the surface of the substrate with a fluid that contains the proteinaceous cell-binding agent to be deposited on the substrate surface. The fluid may be a buffer or other fluid compatible with the proteinaceous cell-binding agent. In some instances, the fluid containing the proteinaceous cell-binding agent is contacted with substantially the entire surface of the substrate. As described above, in some cases, the proteinaceous cell-binding agent may only become attached to the cell-binding surface on the hydrophilic regions of the surface of the substrate, and may not significantly attach to the hydrophobic regions of the surface of the substrate. In other embodiments, the fluid containing the proteinaceous cell-binding agent is contacted with only certain regions on the surface of the substrate. For instance, the fluid containing the proteinaceous cell-binding agent may only be deposited on the hydrophilic regions of the surface of the substrate.

The proteinaceous cell-binding agent may be provided in various concentrations. For example, the proteinaceous cell-binding agent may be provided in a solution where the concentration of the proteinaceous cell-binding agent is 1 μg/mL or more, such as 5 μg/mL or more, or 10 μg/mL or more, including 15 μg/mL or more, or 20 μg/mL or more, or 25 μg/mL or more, or 30 μg/mL or more, or 35 μg/mL or more, or 40 μg/mL or more, or 45 μg/mL or more, or 50 μg/mL or more. In certain embodiments, the concentration of the proteinaceous cell-binding agent is 25 μg/mL.

The proteinaceous cell-binding agent may be contacted with the cell-binding surface for a period of time sufficient to allow the proteinaceous cell-binding agent to attach to the cell-binding surface. For instance, the proteinaceous cell-binding agent may be contacted with the cell-binding surface for a period of time such that the proteinaceous cell-binding agent attaches to the cell-binding surface and is not significantly removed from the cell-binding surface by subsequent washing of the cell culture substrate. In certain cases, the period of time sufficient to attach the proteinaceous cell-binding agent to the cell-binding surface is 24 hours or less, including 20 hours or less, such as 16 hours or less, or 12 hours or less, or 10 hours or less, or 8 hours or less, or 6 hours or less, or 4 hours or less, or 2 hours or less. In certain embodiments, the period of time sufficient to attach the proteinaceous cell-binding agent to the cell-binding surface is 8 hours or less. In certain embodiment, the step of contacting the surface of the substrate with the proteinaceous cell-binding agent may be performed at a temperature less than ambient room temperature. For example, the step of contacting the surface of the substrate with the proteinaceous cell-binding agent may be performed at a temperature of 25° C. or less, such as 20° C. or less, including 15° C. or less, or 10° C. or less, or 5° C. or less. In some cases, the step of contacting the surface of the substrate with the proteinaceous cell-binding agent is performed at 4° C.

In certain embodiments, the proteinaceous cell-binding agent is contacted to the substrate surface at substantially the same time as the surfactant. For instance, the proteinaceous cell-binding agent and the surfactant may be mixed together prior to contacting the mixture of the proteinaceous cell-binding agent and the surfactant to the substrate surface. In other embodiments, separate solutions of the proteinaceous cell-binding agent and the surfactant are contacted to the substrate surface at substantially the same time (e.g., substantially simultaneously). In yet other embodiments, the proteinaceous cell-binding agent and the surfactant are contacted to the substrate surface sequentially. For instance, the surfactant may be contacted to the substrate surface first, thus forming the cell-binding surface, as described above. Subsequently, the proteinaceous cell-binding agent may be contacted to the formed cell-binding surface as described above.

In certain instances, the method further includes removing unbound proteinaceous cell-binding agent from the cell culture substrate. For example, the method may include washing unbound proteinaceous cell-binding agent away from the substrate surface. The washing may include washing the substrate surface with a solution, such as a buffer solution (e.g., phosphate buffered saline). In some cases, the step of washing unbound proteinaceous cell-binding agent from the cell culture substrate is performed after contacting the substrate surface with the proteinaceous cell-binding agent for a sufficient amount of time to allow the proteinaceous cell-binding agent to attach to the cell-binding surface, but before the subsequent step of contacting the substrate surface with the cells. In certain embodiments, washing the unbound proteinaceous cell-binding agent from the substrate surface does not significantly remove proteinaceous cell-binding agent attached to the cell-binding surface on the hydrophilic regions of the substrate surface. One or more washing steps may be performed as desired.

Utility

The cell culture substrates, kits and methods of making the cell culture substrates find use in a variety of different applications where it is desirable to culture cells on a substrate. The cell culture substrates, kits and methods find use in cell culture applications for any type of cell to be cultured. For example, the cell culture substrates, kits and methods may be used to culture cells, such as, but not limited to, human and animal cell lines (e.g., aorta endothelial cells, neuron stem cells, mesenchymal stem cells, and the like).

The methods described herein additionally find use in producing patterned substrates for cell culturing, for example on substrates such as, but not limited to, glass, polystyrene, and other types of cell culture substrates. The methods also find use in the production of cell culture substrates where clean-room facilities and harsh chemicals that may be harmful to cells are not required. For example, embodiments of certain methods for the production of the cell culture substrates may not use toxic compounds or chemicals that may inhibit or prevent cell growth and/or maintenance on the cell culture substrate. The methods described herein find use in the efficient production of patterned cell culture substrates at a low cost. In addition, the methods described herein find use in the production of cell culture substrates that are stable during long-term storage of the cell culture substrates before use.

Embodiments of the cell culture substrates, kits and methods also find use in long-term patterned cell culture and study, for example, in the study of cell differentiation over time. The presently disclosed cell culture substrates and kits provide cell culture substrates that are biocompatible and chemically stable, such that patterned cell cultures may be maintained on the cell culture substrates for extended periods of time, e.g., 1 week or more, such as 2 weeks or more, including 3 weeks or more, or 4 weeks or more, or several months or more (e.g., 2 months, 3 months, 4 months, 5 months, 6 months, or 9 months, or 12 months or more).

The cell culture substrates, kits and methods of making the cell culture substrates also find use in biomedical devices. For example, single-cell culture substrates can be used in biomedical devices for single cell detection, 2D and 3D tissue printing for artificial organ reconstruction where a wide range of cell culture pattern shapes and sizes may be desired, and the like. In addition, embodiments of the cell culture substrates, kits and methods also find use in tissue regeneration applications, where single-cell cultures are desired. Single-cell culture substrates also find use in applications where it is desirable to minimize cell-to-cell interactions. For instance, single cell culture substrates may be used to study individual cell characteristics, for testing the effects of compounds (e.g., drugs, etc.) on individual cells, and the like.

Kits

Also provided are kits for use in practicing the methods. The kits include a cell culture substrate, e.g., as described above. In certain embodiments, the kits also include a proteinaceous cell-binding agent, such as, but not limited to, fibronectin, serum, and the like. In some instances, the kits may include cells for applying to the cell culture substrate. The kit components may be present in separate containers. Alternatively, the components may be present as a packaged element that includes one or more kit components, such as those described above.

In certain embodiments, the kits include additional reagents and/or solutions. The reagents and/or solutions may be used in the cell culture, for example, phosphate-buffered saline (PBS), a surfactant, an antibiotic, a cell culture medium, a fluorescent labeling solution, and the like. As described above, the additional reagents and/or solutions may be provided in separate containers, or as components in a packaged kit.

In addition to above-mentioned components, the kits may further include instructions for using the components of the kit to practice the methods. The instructions for practicing the methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging), etc. In other embodiments, the instructions are present as an electronically stored data file present on a suitable computer readable storage medium, e.g., CD, DVD, Blu-ray, flash memory, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded.

The following examples are put forth so as to provide those of ordinary skill in the art with a complete disclosure and description of how to make and use the embodiments of the invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLES

### Example 1

Surface patterning for single-cell culture was accomplished by combining plasma-assisted surface chemical modification, soft lithography, and protein-induced surface activation. Hydrophilic patterns were produced on Parylene C films deposited on glass substrates by oxygen plasma treatment through the windows of polydimethylsiloxane shadow masks. After incubation first with Pluronic F108 solution and then serum medium overnight, surfaces seeded with mesenchymal stem cells in serum medium resulted in single-cell patterning. The experiments provided a method of surface patterning to produce an array of single-cell cultures on a substrate.

A method of single-cell patterning was developed by combining Parylene C film deposition, oxygen plasma treatment through the windows of polydimethylsiloxane (PDMS) shadow masks, incubation with a Pluronic F108 solution, and surface activation by incubation with serum medium. The efficacy of the present method was demonstrated by experiments showing the preferential attachment of single cells on the hydrophilic surface areas of chemically patterned Parylene C films.

Experimental

Polydimethylsiloxane (PDMS) shadow masks were fabricated as follows. Arrays of microposts of 50 μm in height and 200 μm in lateral spacing were fabricated on a p-type Si(100) wafer using SU-8 2050 photoresist (MicroChem, Newton, Mass.) to obtain a master wafer, which was then exposed to perfluoro-1,1,2,2-tetrahydrooctyl-trichlorosilane (United Chemical Technology, Bristol, Pa.) vapor overnight in a desiccator to prevent adhesion of the PDMS mask to the substrate. Subsequently, the master wafer was spin coated with a 30 μm-thick PDMS film, using a 10:1 mixture of Sylgard 184 silicone elastomer kit (Dow Corning, Midland, Mich.), which was cured at 65° C. for 4 hr. The PDMS membrane mask was then cut into 1.7×1.7 cm² pieces, each having window arrays of specific shape and size, which were carefully peeled off from the master wafer using a pair of tweezers and a piece of a glass slide.

Parylene C films of 0.5 μm in thickness were deposited on glass substrates using a commercial coating system (PDS 2010 LABCOTER 2, Indianapolis, Ind.). To create hydrophilic surface patterns, the PDMS mask was first placed on the surface of the Parylene C film conformably, and the whole surface was treated with oxygen plasma for 1 min in a plasma etch system (Plasma Prep, SPI supplies/Structure Probe, West Chester, Pa.). Film areas exposed to the plasma became hydrophilic (contact angle≈0°), while areas covered by the mask maintained their hydrophobic character (contact angle≈90°). Subsequently, the PDMS mask was removed and the patterned Parylene C surface was sterilized with ultraviolet light for at least 30 min and then incubated either with a solution of Pluronic F108 copolymer (BASF, Mount Olive, N.J.) in phosphate-buffered saline (PBS) or a mixture of Pluronic F108 solution and fibronectin (Sigma-Aldrich, St. Louis, Mo.) in PBS. After overnight incubation at 4° C., the patterned surfaces were first washed with PBS three times and then seeded with mesenchymal stem cells (MSCs) in serum medium consisting of Dulbecco's modified Eagle's medium (DMEM), 10% fetal bovine serum, and 1% penicillin streptomycin. After incubation for 1 hr in 5% CO₂ at 37° C., floating MSCs were washed away and fresh serum medium was added. Fluorescein isothiocyanate (FITC)-collagen type I (Sigma-Aldrich, St. Louis, Mo.) was used to examine protein adsorption on the chemically patterned Parylene C film surfaces.

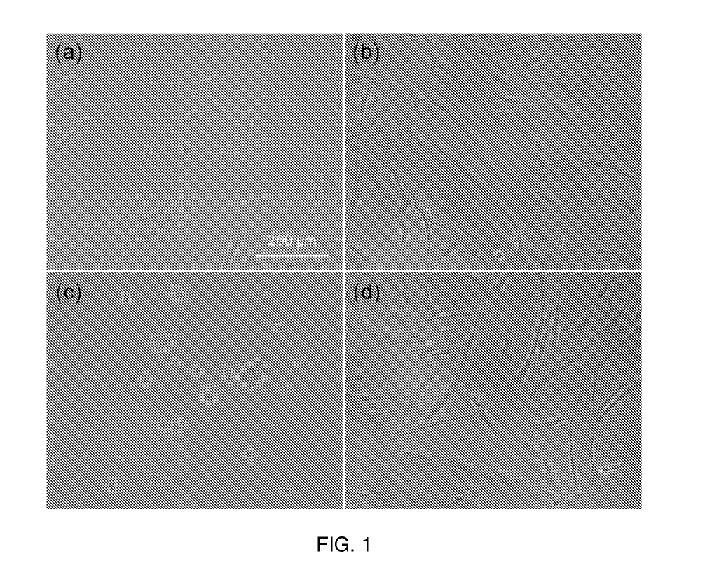

To examine if the adsorption of Pluronic molecules on the hydrophobic and hydrophilic Parylene C surfaces affected MSC attachment, untreated and oxygen plasma-treated (1 min) glass coverslips coated with Parylene C films were incubated with 0.1% Pluronic F108 in PBS for 1 hr. After washing three times with PBS, the surfaces were seeded with MSCs in serum medium. Overnight incubation resulted in MSC attachment and spreading on the plasma-treated (hydrophilic) surface [FIG. 1(d)] but not the untreated (hydrophobic) surface where the cells were still floating [FIG. 1(c)]. However, MSCs attached on both control samples, i.e., untreated [FIG. 1(a)] and plasma-treated [FIG. 1(b)] Parylene C surfaces that had not been incubated with Pluronic solution. This difference in MSC attachment indicated that the oxygen plasma changed the surface chemical behavior of Parylene C from hydrophobic to hydrophilic, affecting the adsorption of Pluronic molecules. MSCs did not attach on the hydrophobic areas because the brush-like configuration of the adsorbed Pluronic molecules repelled protein adsorption and cell attachment. However, because this brush-like molecular arrangement was not thermodynamically favored on the hydrophilic areas, protein adsorption and, in turn, MSC attachment on these areas was not prevented.

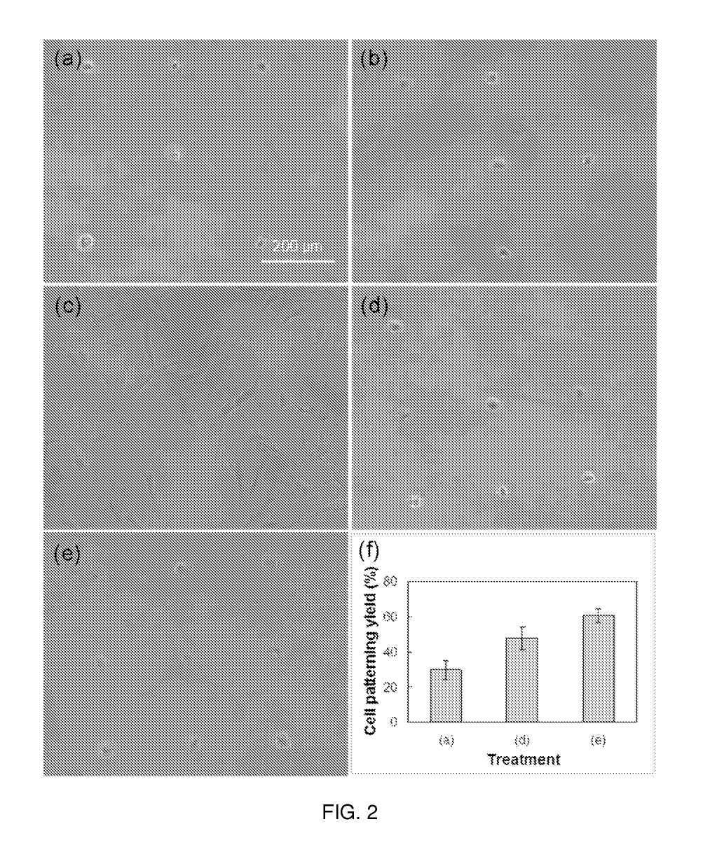

These differences in cell attachment between hydrophilic and hydrophobic Parylene C surfaces covered with Pluronic molecules provided an effective means of surface patterning for single-cell culture. MSC attachment to the hydrophilic areas of a patterned Parylene C surface incubated with 0.1% Pluronic solution overnight was observed [FIG. 2(b)]. Similarly, with a decreased Pluronic concentration of 0.01%, single cell attachment was observed on the hydrophilic areas [FIG. 2(a)]. To enhance cell attachment on the hydrophilic areas, fibronectin was added to the Pluronic solution. Pluronic concentration was increased to at least 0.1% (for the concentration range of this study) to prevent fibronectin adsorption and cell attachment on the hydrophobic areas. For a low Pluronic concentration (0.01%), MSCs attached on both hydrophilic and hydrophobic areas [FIG. 2(c)] without resulting in single-cell patterning. Increasing the Pluronic concentration to 0.1% resulted in single-cell patterning [FIG. 2(d)].

An additional method to enhance cell attachment included a surface activation step with serum medium, which was added before cell seeding. After incubating a chemically patterned Parylene C surface with 0.01% Pluronic solution for 1 hr and washing three times with PBS, the whole surface was incubated with serum medium overnight at 37° C. to activate the hydrophilic areas before cell seeding. MSCs attached and spread on the serum-activated areas after incubation for 1 hr [FIG. 2(e)], producing a higher yield of single-cell patterning than patterned Parylene C surfaces that had not been previously activated with serum, as shown by the cell patterning yield data of FIG. 2(f). The data represent the percentages of single-cell pattern areas after washing away all floating cells. In some cases, the use of a low cell density for promoting the attachment of a single cell on each pattern area resulted in a yield of less than 100%.

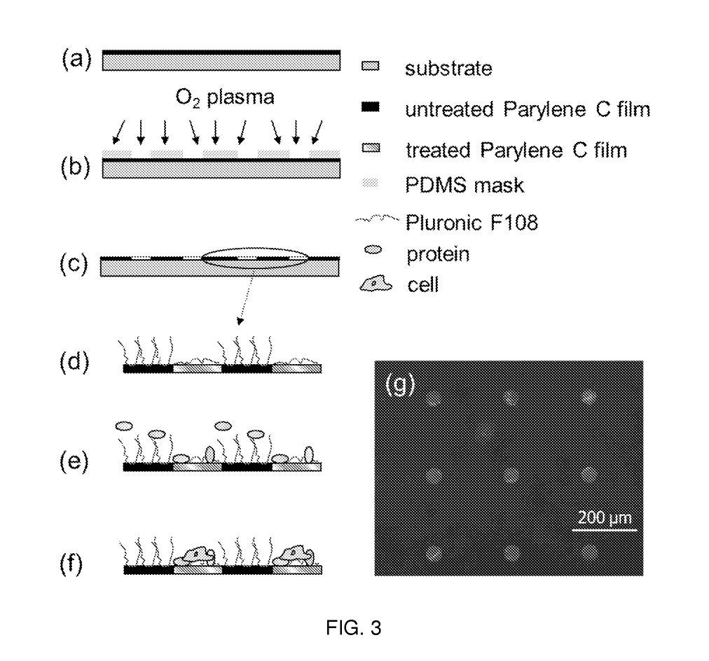

Adsorption of the amphiphilic Pluronic molecules on the patterned Parylene C surfaces occurred by the preferential attachment of the hydrophobic poly-propylene oxide (PPO) and hydrophilic poly-ethylene oxide (PEO) segments to the hydrophobic and hydrophilic Parylene C areas, respectively [FIG. 3(d)]. Without being limited to any particular theory, differences in MSC attachment may be due to protein adsorption on the hydrophilic areas covered with Pluronic molecules lying flat on the surface [FIG. 3(e)] but not on the hydrophobic areas because of steric repulsion of the freely sawing

PEO segments. Gradual modification of the hydrophilic areas by serum proteins played a role in the preferential attachment of MSCs on the hydrophilic areas of the activated Parylene C surface [FIG. 3(f)], resulting in a high yield of single-cell patterning [FIG. 2(e)].

Preferential protein adsorption on the hydrophilic areas covered with Pluronic molecules was confirmed by FITC-collagen adsorption tests. After incubation with 0.01% Pluronic solution for 1 hr, the patterned Parylene C surface was first washed with PBS three times and then incubated with FITC-collagen solution (200 μg/mL) overnight at room temperature. Fluorescence photographs (obtained after washing the incubated surface with PBS three times) showed significantly more FITC-collagen adsorption on the pattern areas [FIG. 3(g)], confirming that incubation with serum medium resulted in the activation of the hydrophilic pattern areas. Cultures in serum medium showed that the surface patterns were stable for more than two weeks.

Results

A surface patterning method for single-cell culture was developed in this study. The method uses Parylene C film deposition and surface chemical modification by oxygen plasma treatment through the windows of a PDMS shadow mask, and relies on the effect of surface hydrophilicity on the adsorption configuration of Pluronic molecules and surface activation by serum proteins. Parylene C is a coating material that can be applied to other substrate materials that cannot be easily patterned with traditional microcontact printing methods.

### Example 2

Surface chemical patterning of polystyrene dishes for long-term single-cell culture was accomplished by oxygen plasma treatment through the windows of a polydimethylsiloxane membrane mask that produced hydrophilic surface areas of different shapes and sizes, followed by overnight incubation with either Pluronic F108 solution or a mixture of Pluronic F108 solution and fibronectin. Selective cell attachment on pattern areas of polystyrene dishes was investigated in light of cell seeding experiments and X-ray photoelectron spectroscopy (XPS) measurements. Activation of hydrophilic areas of patterned polystyrene surfaces by serum proteins in the culture medium was conducive to cell attachment on the pattern areas of dishes incubated with only Pluronic solution. Preferential adsorption of fibronectin on hydrophilic pattern areas enhanced selective cell attachment on patterned dishes incubated with a mixture of Pluronic solution and fibronectin. Cell culture experiments demonstrated an effect of surface patterning on both cell and nucleus shape and confirmed the long-term (e.g., 2 weeks or more) stability of the produced single-cell patterns in serum medium.

Pluronic is a copolymer of polyethylene oxide (PEO) and polypropylene oxide (PPO) segments, which can be adsorbed on some hydrophobic surfaces to prevent protein adsorption and cell attachment. Pluronic adsorption occurs by the attachment of PPO segments to the hydrophobic surface, with the PEO segments extending away from the surface, forming a brush-like non-fouling film. However, this configuration is not favored on hydrophilic surfaces, where the PEO segments lay flat on the surface. Therefore, the chemical behavior of Pluronic molecules adsorbed on hydrophobic and hydrophilic surfaces should differ significantly. For example, competitive adsorption of Pluronic F68 and fibronectin molecules on the hydrophobic and hydrophilic areas of plasma-modified polystyrene surfaces results in cell attachment only to the hydrophilic polystyrene areas.

Traditional photolithography used in previous studies to produce hydrophilic surface patterns on polystyrene cannot be directly applied to cell culture dishes because it requires a clean-room facility and chemicals that could be harmful to cells. To overcome this problem, a dry lithography method that can be directly applied to polystyrene Petri dishes was developed according to embodiments of the present disclosure. The objectives of the experiments presented herein were to identify the plasma treatment conditions and incubation solution of a surface patterning method for selective cell attachment, analyze changes in surface chemistry induced by the patterning process, explore the long-term stability of the patterned surfaces in culture medium, and examine the effect of surface patterning on both cell and nucleus shape. In certain embodiments, the method uses (a) oxygen plasma treatment through the windows of a polydimethylsiloxane (PDMS) membrane mask to form hydrophilic patterns on regular culture dishes and (b) surface hydrophilicity resulting in selective molecular (Pluronic, fibronectin, or serum proteins) adsorption to produce chemical patterns for single-cell culture.

Patterned dishes were incubated either with Pluronic F108 solution or a mixture of Pluronic F108 solution and fibronectin. Cell culture experiments and X-ray photoelectron spectroscopy (XPS) studies elucidated the underlying mechanisms of selective cell attachment. In addition, long-term cell culture experiments demonstrated an effect of surface patterning on the cell and nucleus shapes and confirmed the stability of the produced patterns in culture medium.

Experimental Procedures

The PDMS membrane mask was fabricated as follows. Micropost arrays of ˜50 μm height and 200 μm lateral spacing were fabricated on a p-type Si(100) wafer using

SU-8 2050 photoresist (MicroChem, Newton, Mass.) to obtain a master wafer. Before coating with PDMS, the master wafer was exposed to perfluoro-1,1,2,2-tetrahydrooctyltrichlorosilane (United Chemical Technology, Bristol, Pa.) vapor overnight in a desiccator to prevent PDMS from adhering to the master wafer. Then, the master wafer was spin coated with a mixture (10:1) of Sylgard 184 silicone elastomer kit (Dow Corning, Midland, Mich.) to produce a 30 μm-thick PDMS film which was cured at 65° C. in 4 hr. The PDMS membrane mask was cut into 1.7×1.7 cm² pieces, each having window arrays of different shapes and sizes, which were carefully peeled off from the master wafer by a pair of tweezers and a piece of glass slide. PDMS masks of window areas equal to 2000 μm² and shape index (SI) equal to 1.0, 0.5, 0.25, and 0.1 (SI=4πA/P², where A and P are the projected area and the perimeter of a pattern area, respectively) were used in cell culture experiments. Shape indexes of 1 and 0 correspond to circular and linear shapes, respectively.

Pluronic F108 (BASF, Mount Olive, N.J.) powder was dissolved in phosphate buffer saline (PBS) to produce a solution of 1% (wt/vol) Pluronic concentration. After overnight storage at 4° C., the solution was passed through a filter of 0.2 μm average pore size to obtain a sterilized stock. Sterilized polystyrene (PS) Petri dishes (BD Falcon, Franklin Lakes, N.J.) were used in their as-received condition. To generate hydrophilic surface patterns, the PDMS mask was conformably placed onto PS dishes and entire dish surfaces were exposed to oxygen plasma for 1 min in a plasma-etch system (Plasma Prep II, SPI supplies/Structure Probe, West Chester, Pa.). Dish areas exposed to the plasma acquired hydrophilic characteristics (contact angle≈0°), whereas areas covered by the PDMS mask maintained their hydrophobic behavior (contact angle≈80°. After the PDMS mask was removed, the patterned dishes were first sterilized with ultraviolet light for at least 30 min and then incubated with either Pluronic F108 solution or Pluronic F108 solution mixed with fibronectin (Sigma-Aldrich, St. Louis, Mo.) for surface blocking. Following overnight incubation at 4° C., the dishes were washed three times with PBS and seeded with cells.

Bone marrow mesenchymal stem cells (MSCs) were used in cell culture experiments. MSCs were seeded with either serum-free medium or serum-containing culture medium consisting of Dulbecco's modified Eagle's medium (DMEM), 10% fetal bovine serum (FBS), and 1% penicillin streptomycin. After 1-hr incubation with 5% CO₂ at 37° C., floating cells were washed away and fresh serum medium was added. Following incubation for 2 weeks, MSCs were fixed with 4% paraformaldehyde (PFA), permeabilized with 0.5% Triton X-100, and cell actin and nucleus were stained with Alexa-Phalloidin 488 and 4′,6-diamidino-2-phenylindole (DAPI), respectively. Phase-contrast pictures of fixed MSCs were obtained with an inverted microscope (TE 300, Nikon, Melville, N.Y.), and fluorescence photographs of stained MSCs were obtained with an upright microscope (Zeiss HAL 100, Carl Zeiss Microlmaging, Thornwood, N.Y.). Cell and nuclei areas and shape indexes were calculated from fluorescence images of cell actin and nucleus. Cell and nucleus boundaries were outlined with software (Scion IMAGE, Fredrick, Md.). Measured cell spreading areas and perimeters and nuclei projection areas and perimeters were used to calculate the cell shape index (CSI) and nucleus shape index (NSI).

Pluronic and fibronectin adsorption on hydrophilic and hydrophobic pattern areas of PS dishes was investigated by XPS. PS samples cut from the bottom of cell culture dishes were partially covered with a PDMS membrane mask and then exposed to oxygen plasma. Partially plasma-treated PS samples were then incubated with PBS, Pluronic F108 solution, or Pluronic F108 mixed with fibronectin overnight, washed three times with PBS, and dried in air prior to XPS analysis. A spectrometer without charge neutralization or monochromator (Perkin-Elmer PHI 5400 ESCA) equipped with Al-Kα X-ray source of photon energy equal to 1486.6 eV was used in all XPS experiments. The take-off angle relative to the analyzer axis was fixed at 54.7°. During spectral acquisition, the pressure in the main chamber was maintained at ˜10⁻⁷ Torr. Survey spectra were acquired in the binding energy range of 0-1100 eV with pass energy of 178.95 eV. High-resolution XPS spectra of C1s, N1s, and O1s core level peaks were collected with pass energy of 35.75 eV. To compensate for surface charging effects, the C—H peak at 285.0 eV was used as a reference. Atomic nitrogen concentration (determined from the N1s core level peak after Shirley background subtraction) was used to study fibronectin adsorption on various sample surfaces. For statistical analysis, XPS data were calculated as averages of four measurements acquired from two different surface regions of two samples with identical treatment histories.

Results and Discussion

The dependence of cell attachment on the adsorption configuration of Pluronic molecules at hydrophobic and hydrophilic PS surfaces was studied by seeding MSCs in serum medium on three different types of dishes: (a) untreated (control), (b) incubated with 1% Pluronic solution for 1 hr, and (c) treated with oxygen plasma, incubated with 1% Pluronic solution for 1 hr, and, finally, washed three times with PBS before cell seeding. While MSCs attached on the untreated dish and to less extent on the oxygen plasma-treated dish incubated with 1% Pluronic solution, they did not attach on the untreated dish incubated with 1% Pluronic solution. After overnight incubation, floating MSCs were collected and reseeded on an untreated dish. These cells were observed to attach and spread well on the untreated dish surface, indicating that previously mentioned differences in cell attachment were not due to possible cytotoxicity effects of Pluronic molecules. Thus, the observed differences in MSC attachment was attributed to Pluronic adsorption on the untreated PS surface in a brush-like non-fouling molecular configuration, as opposed to the plasma-treated dish on which the Pluronic molecules laid flat on the surface. This finding also suggested that, in the presence of serum, MSCs attached to the hydrophilic PS surface covered by Pluronic molecules.

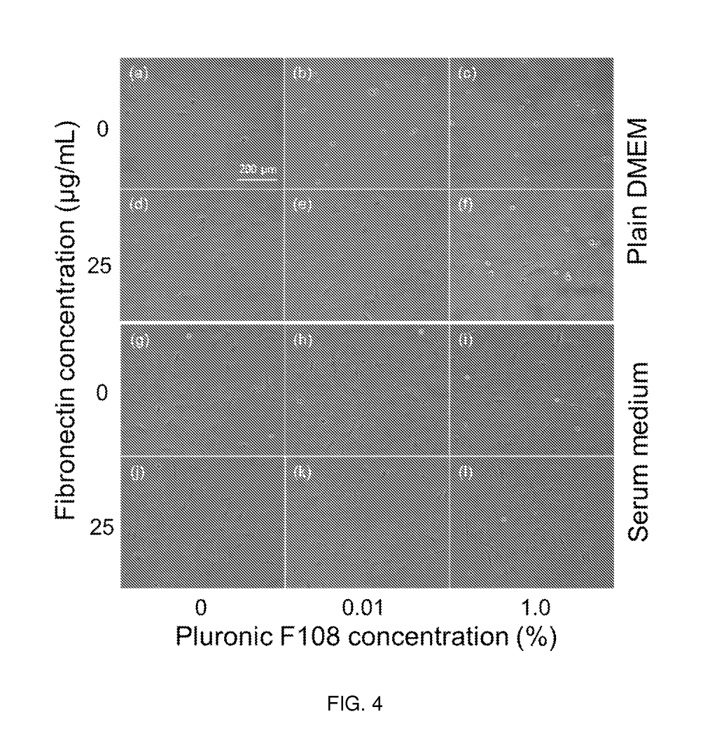

To examine how Pluronic adsorption on hydrophilic PS surfaces affected cell attachment, oxygen plasma-modified dishes were incubated with six different solutions overnight, i.e., PBS, 0.01% Pluronic F108 solution, 1% Pluronic F108 solution, and each of the above solutions mixed with 25 μg/mL of fibronectin. After washing the PS surfaces three times with PBS, MSCs were seeded and incubated with either plain DMEM or serum medium for 4 hr before they were examined under the microscope. FIG. 4 shows representative images from these experiments. MSC incubation with plain DMEM did not result in cell attachment on the surfaces incubated with only Pluronic solution even after 4 hr [FIGS. 4(b) and 4(c)]. However, MSCs attached and spread (though to a different degree) on all other surfaces [FIGS. 4(a) and 4(d)-4(f)], including the surface incubated with only PBS [FIG. 4(a)]. Although Pluronic adsorption on the hydrophilic PS surface did not yield a non-fouling conformation, in the absence of serum it prevented cell attachment [FIGS. 4(b) and 4(c)]. However, Pluronic adsorption on the hydrophilic PS surface did not inhibit fibronectin adsorption. Thus, the addition of fibronectin in the Pluronic solution was conducive to cell attachment, depending on the Pluronic concentration. For 25 μg/mL of fibronectin, increasing the Pluronic concentration from 0.01% [FIG. 4(e)] to 1% [FIG. 4(f)] increased the number of floating cells, indicating less fibronectin adsorption on the hydrophilic PS surface covered with more Pluronic molecules [FIG. 4(f)].

For MSCs incubated with serum medium, differences in cell attachment were only observed in the early incubation stage. MSCs attached rapidly to all surfaces in less than 1 hr, except for the surfaces incubated with only Pluronic solution. However, no difference in cell attachment could be discerned after incubation in serum medium for 4 hr [FIGS. 4(g)-4(l)]. This finding suggested that serum proteins from the culture medium modified or activated the hydrophilic PS surfaces covered by Pluronic molecules, and this activation process was beneficial to cell attachment. Because of the relatively slow modification/activation process, MSC attachment on dish surfaces treated with only Pluronic did not occur during the initial stage of incubation. This is in agreement with the longer time for cell attachment observed with higher Pluronic concentration and is attributed to coverage of the hydrophilic PS surface by more Pluronic molecules that slowed down the activation process.

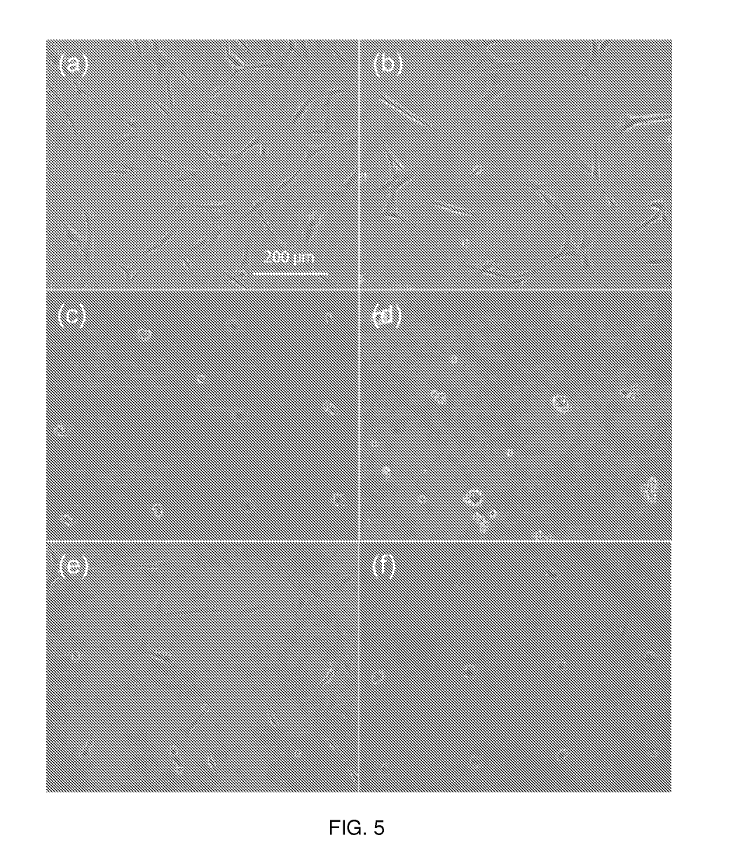

For single-cell patterning, PS dishes with hydrophilic patterns (produced by oxygen plasma treatment through the windows of PDMS masks) were incubated with different solutions before seeding with MSCs in serum medium overnight. An untreated dish was used as control [FIG. 5(a)]. Similar to the control sample in FIG. 5(a), MSCs attached everywhere on the patterned dish incubated with PBS [FIG. 5(b)]. This suggested that high selectivity in cell attachment cannot be accomplished through modifications of the surface hydrophilicity alone. Dishes treated with Pluronic solutions of concentrations in the range of 0.001%-1% (with or without the addition of 25 μg/mL of fibronectin) were seeded with MSCs in serum medium. Incubation with only low concentration (0.01% or less) Pluronic resulted in rapid single-cell patterning [FIG. 5(c)]. Increasing the Pluronic concentration to 1% not only increased the time for cell attachment but also reduced the number of attached cells significantly [FIG. 5(d)]. The addition of 25 μg/mL of fibronectin in the Pluronic solution of concentration equal to 0.01% yielded random cell attachment [FIG. 5(e)], suggesting that fibronectin adsorbed on both hydrophilic and hydrophobic dish areas. However, increasing the Pluronic concentration to a higher level (i.e., ≧0.1%) restored single-cell patterning [FIG. 5(f)]. These results indicated that, for a given fibronectin concentration, the Pluronic concentration must be above a threshold to prevent fibronectin adsorption on untreated (hydrophobic) PS, which affected single-cell patterning. Thus, although fibronectin promoted MSC attachment on hydrophilic pattern areas, the Pluronic concentration had to be increased to achieve single-cell patterning. For cell seeding in serum medium, the time for cell attachment on the hydrophilic areas of the patterned dish incubated with 0.01% Pluronic solution was comparable to that of the patterned dish incubated with 0.1% Pluronic solution and 25 μg/mL of fibronectin.

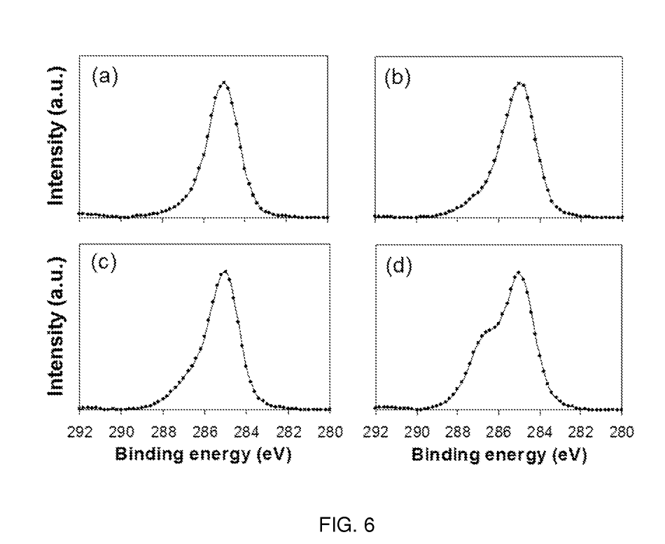

Pluronic and fibronectin adsorption on untreated and oxygen plasma-treated dishes was further examined with the XPS. Representative C1s spectra of PS surfaces subjected to different treatments are shown in FIG. 6. The C1s peak of the oxygen plasma-treated PS surface [FIG. 6(b)] differs slightly from that of the untreated surface [FIG. 6(a)]. The small change in the C1s peak is attributed to oxygen functionalities on the plasma-treated PS surface. Incubation with 1% Pluronic solution did not yield a detectable effect on the C1s peak of untreated PS [FIG. 6(c)], due to Pluronic desorption during drying. However, incubation with Pluronic significantly changed the C1s peak of the oxygen plasma-treated PS surface [FIG. 6(d)], implying relatively stable adsorption of Pluronic molecules. The former observations are supported by XPS results of the O/C ratio. For PS surfaces with C1s spectra shown in FIGS. 6(a)-6(d), the corresponding O/C ratio was found equal to 0.26, 0.33, 0.35, and 0.50, respectively. These results confirmed that Pluronic molecules adsorbed on the hydrophilic PS areas, affected cell attachment despite the fact that molecular assembly was not of non-fouling configuration. The nitrogen concentration of hydrophobic and hydrophilic PS surfaces incubated with 0.01% Pluronic overnight and then with 10% FBS medium for 1 hr was also examined with the XPS after washing the samples with PBS. The nitrogen concentration of hydrophobic and hydrophilic PS surfaces was found equal to 6.05 and 8.85 at %, respectively, implying that hydrophilic PS surfaces exhibited higher protein concentrations than hydrophobic PS surfaces. This result confirmed the attachment of cells on the hydrophilic PS surface activated with serum medium.

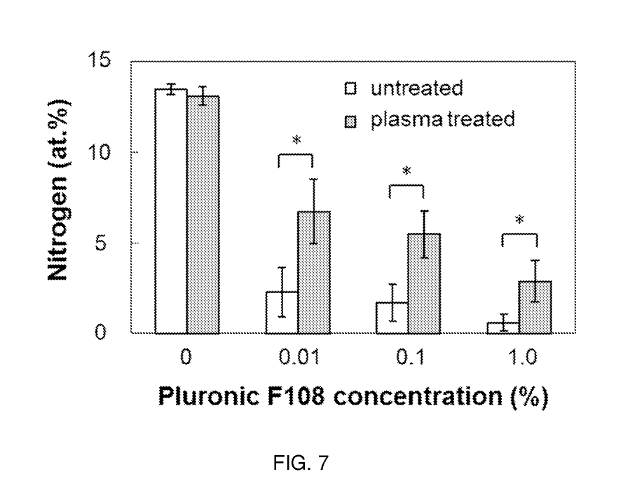

FIG. 7 shows the nitrogen content of hydrophobic and hydrophilic PS surfaces incubated with a mixture of Pluronic solution and 25 μg/mL of fibronectin for Pluronic concentration in the range of 0-1%. The significant decrease in nitrogen content of the PS surfaces incubated with high-concentration Pluronic solutions, especially untreated PS, indicated that Pluronic adsorption blocked fibronectin adsorption. Although fibronectin adsorption on the hydrophilic PS surfaces also decreased in the presence of Pluronic, the significantly higher nitrogen concentration of the hydrophilic PS surfaces explained the selective attachment of cells on hydrophilic pattern areas. The decrease in Pluronic concentration yielded an increase in fibronectin adsorption on both hydrophilic and hydrophobic PS surfaces. This trend was observed in the experiments with MSCs incubated with different Pluronic solutions that contained 25 μg/mL of fibronectin. Although Pluronic adsorption significantly suppressed fibronectin adsorption on untreated PS surfaces, fibronectin also adsorbed on the hydrophobic areas of the patterned dish surface. Therefore, for single-cell patterning, the Pluronic concentration must be above a threshold to prevent excessive fibronectin adsorption on hydrophobic PS areas.

Experiments were performed that included MSCs seeded on patterned dishes after incubation with a mixture of 0.01% Pluronic solution and 25 μg/mL of fibronectin for more than 2 days. While initially MSCs spread outside the pattern areas, they retracted later within the pattern areas. This finding suggested that prolonged incubation was conducive to cell sensing of chemical differences between hydrophilic and hydrophobic pattern areas, resulting in cell migration back to the hydrophilic pattern areas with higher fibronectin concentration.

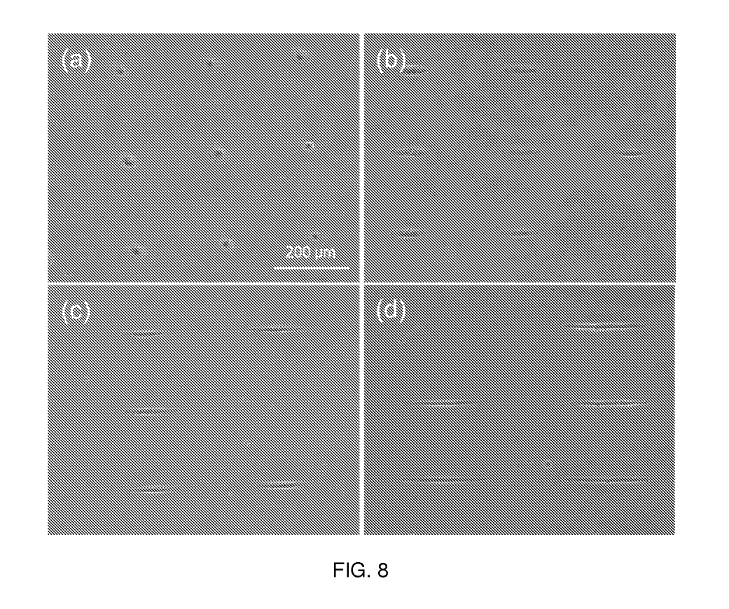

To examine the long-term stability of surface patterning, MSCs were seeded on patterned dishes incubated either with a relatively low-concentration Pluronic solution or a high-concentration Pluronic solution that contained 25 μg/mL of fibronectin. All patterns remained stable even after incubation in serum medium for more than 2 weeks. FIG. 8 shows MSCs seeded on patterned PS surfaces that were treated with a 1% Pluronic solution with 25 μg/mL of fibronectin after incubation with serum medium for 2 weeks. These MSCs were confirmed to be alive by cell live/dead assay results (not shown here). Circular [FIG. 8(a)] and elliptical [FIGS. 8(b)-8(d)] patterns on the dish surfaces were occupied by single MSCs that had spread out to fully cover only the hydrophilic areas of high fibronectin concentration.

Similar results were also obtained from single-cell culture experiments with neuron stem cells (NSCs) and bovine aorta endothelial cells (BAECs) (data not shown). The long-term stability of single-cell patterns on PS dish surfaces produced by the present method was also observed in cell culture experiments with NSCs and BAECs.

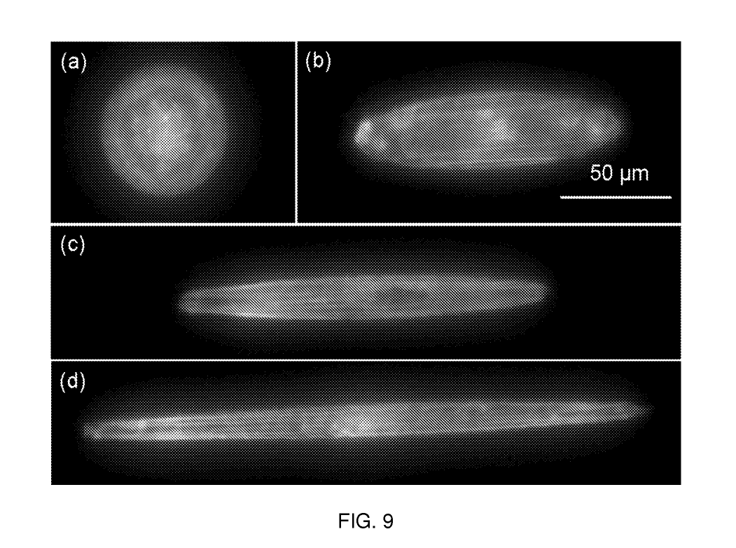

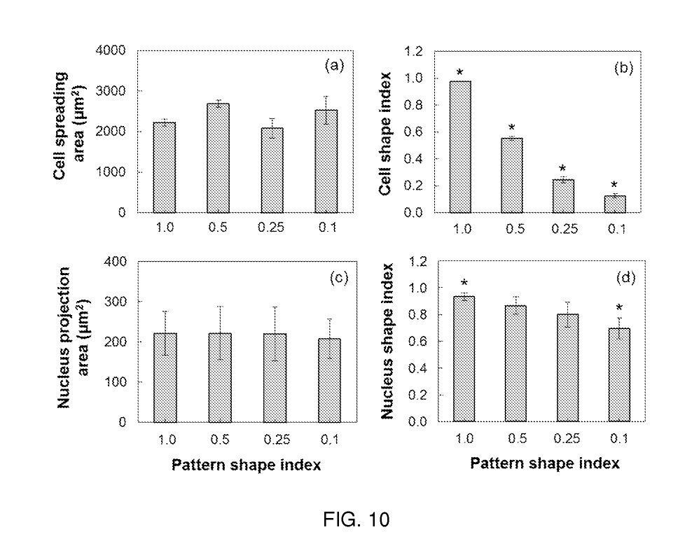

FIG. 9 shows fluorescence pictures of MSCs obtained after 2 weeks of incubation in serum medium that reveal the cell nuclei and actin structure. Circular MSCs demonstrated fairly round nuclei and actin alignment along the radial direction and also around the nucleus [FIG. 9(a)], whereas elongated MSCs showed nucleus elongation and actin alignment along the major axis of their elliptical shapes [FIGS. 9(b)-9(d)]. In general, actin remodeling occurred within 1 day of incubation and then stabilized. Cell and nucleus geometry measurements obtained from fluorescence pictures are shown in FIG. 10. Results of the cell spreading area [FIG. 10(a)] and cell shape index (CSI) [FIG. 10(b)] were in good agreement with design parameters. For similar cell spreading area, the nucleus projection area did not significantly change with CSI [FIG. 10(c)]. NSI was affected by CSI variations [FIG. 10(d)], although NSI change was relatively less as compared to the change in CSI.

Since the present patterning method is based on dry lithography and hydrophilicity-controlled surface patterning, it can be used to generate different patterns on the same dish surface. For example, after the formation of a specific hydrophilic pattern on a PS dish, the PDMS mask can be lifted and another mask with windows of different sizes and shapes can be placed conformably on the same dish surface to produce different hydrophilic patterns. Thus, combinations of different single-cell patterns on the same dish surface may be produced without the need to design a new chromium mask, as would be the case in traditional photolithography methods.

Conclusions

A surface patterning method for single-cell culture was developed by combining plasma-assisted surface modification through the windows of PDMS masks to produce hydrophilic and hydrophobic surface areas and overnight incubation with Pluronic solutions of different concentrations, with and without the addition of fibronectin. Compared to other methods, the present method does not require precise control of the patterning process and is effective in producing a wide range of pattern shapes and sizes for single-cell culture. Long-term (e.g., 2 weeks or more) cell culture experiments yielded insight into the effect of surface patterning on the cell and nucleus shapes and confirmed the long-term stability of the produced single-cell patterns in serum medium.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A cell culture substrate comprising:
a substrate comprising a surface having at least one hydrophilic region and at least one hydrophobic region; and
a surfactant layer present on the surface of the substrate and configured to produce a cell-binding surface on the hydrophilic regions of the surface of the substrate.

2. The cell culture substrate of claim 1, wherein the hydrophilic region has a contact angle less than 90°.

3. The cell culture substrate of claim 1, wherein the hydrophobic region has a contact angle greater than 90°.

4. The cell culture substrate of claim 1, wherein the hydrophilic region has a greater amount of oxygen-containing functional groups than the hydrophobic region.

5. The cell culture substrate of claim 1, wherein the substrate comprises a polymeric substrate or a polymeric layer on the surface of a non-polymeric support.

6. The cell culture substrate of claim 5, wherein the polymeric substrate comprises polystyrene.

7. The cell culture substrate of claim 5, wherein the non-polymeric support comprises glass.

8. The cell culture substrate of claim 7, wherein the polymeric layer comprises a poly(p-xylylene) polymer.

9. The cell culture substrate of claim 1, wherein the surfactant layer comprises a copolymer of polyalkylene oxides.

10. The cell culture substrate of claim 1, wherein the surfactant layer is present on the hydrophilic regions and the hydrophobic regions of the surface of the substrate.

11. The cell culture substrate of claim 1, further comprising a proteinaceous cell-binding agent disposed on the cell-binding surface.

12. The cell culture substrate of claim 11, wherein the proteinaceous cell-binding agent comprises fibronectin.

13. The cell culture substrate of claim 1, wherein the proteinaceous cell-binding agent comprises serum.

14. The cell culture substrate of claim 1, further comprising a cell disposed on the cell-binding surface.

15. The cell culture substrate of claim 13, wherein a single cell is disposed on each cell-binding surface.

16. The cell culture substrate of claim 1, wherein the cell culture substrate is configured to provide for stable cells for 1 week or more.

17. The cell culture substrate of claim 1, wherein the surface of the substrate comprises an array of hydrophilic regions in the hydrophobic region.

18. A method for producing a cell culture substrate, the method comprising:
exposing a substrate to a plasma to produce a surface having at least one hydrophilic region and at least one hydrophobic region; and
applying a surfactant layer to the surface of the substrate to produce a cell-binding surface on the hydrophilic regions of the surface of the substrate.

19. The method of claim 18, wherein the exposing the substrate to the plasma comprises:
applying a mask comprising a masked portion and an unmasked portion to the surface of the substrate;
contacting the masked substrate with a plasma to produce a hydrophilic region in the unmasked portion of the substrate; and
removing the mask from the substrate.

20. The method of claim 18, further comprising disposing a cell on the cell-binding surface.

21. The method of claim 20, wherein a single cell is disposed on each cell-binding surface.

22. The method of claim 20, further comprising contacting the cell-binding surface with a proteinaceous cell-binding agent prior to disposing the cell on the cell-binding surface.

23. The method of claim 20, further comprising removing unbound cells from the substrate.

24. The method of claim 18, further comprising depositing a polymeric layer on the surface of the substrate prior to exposing the substrate to the plasma.

25. A kit comprising:
a cell culture substrate comprising:
a substrate comprising a surface having at least one hydrophilic region and at least one hydrophobic region; and
a surfactant layer present on the surface of the substrate and configured to produce a cell-binding surface on the hydrophilic regions of the surface of the substrate; and
a proteinaceous cell-binding agent.
